(19) United States
(12) Patent Application Publication
(10) Pub. No.: US 2023/0310810 A1
(43) Pub. Date: Oct. 5, 2023

(54) SLOTTED TUBING SECURITY PLATFORM

(71) Applicant: Alcon Inc., Fribourg (CH)

(72) Inventors: James Y. Chon, Irvine, CA (US); Joel Cicchella, Tustin, CA (US); Robert Jeffrey Heng, Lake Forest, CA (US); Grace Chuang Liao, Irvine, CA (US); Ashish Sinha, Irvine, CA (US)

(21) Appl. No.: 18/190,191

(22) Filed: Mar. 27, 2023

Related U.S. Application Data
(60) Provisional application No. 63/362,229, filed on Mar. 31, 2022.

(51) Int. Cl. A61M 25/02; A61F 9/007

(52) U.S. Cl. CPC A61M 25/02; A61F 9/007

(57) ABSTRACT

A platform configured to secure a service loop of tubing from a fixed position surgical device. The platform allows for avoidance of taping and re-taping of service loops. Additionally, the platform provides stability to the surgical device so as to avoid unintentional changing of position or orientation of the device. This is of particular benefit for an infusion port during eye surgery and other similar devices where orientation of the installed device aids device performance.

### BACKGROUND

Over the years, many dramatic advancements in the field of eye surgery have taken place. In some eye surgeries, a vitrectomy will be included in at least part of the procedure. Vitrectomy is the removal of some or all of the vitreous humor from a patient's eye. In some cases, where the surgery was limited to removal of clouded vitreous humor, the vitrectomy may constitute the majority of the procedure. However, a vitrectomy may accompany cataract surgery, surgery to repair a retina, to address a macular pucker or a host of other issues.

The vitreous humor itself is a clear gel that may be removed by an elongated probe when inserted through a pre-placed cannula at the eye. More specifically, the probe includes a central channel for removal of the vitreous humor. Further, the cannula provides a structurally supportive conduit strategically located at an offset location at the front of the eye, such as the pars plana. In this way, the probe may be guidingly inserted into the eye in a manner that avoids damage to the patient's lens or cornea.

Unfortunately, removal of the vitreous humor requires greater care than simply applying a vacuum through the channel of the vitrectomy probe. For example, given that vitreous humor is being removed, measures may be taken to ensure that pressure within the eye is maintained. This may include the use of an infusion port. That is, as with the intervention for removal of material from the eye, another pre-placed cannula may be provided to accommodate the influx of fluid to the eye so that a pressure balance is maintained. So, for example, where an eye interior may be at about 30 mm/Hg (millimeters of mercury) in pressure, an infusion port may be utilized to maintain the eye interior, for example, from about 15 mm/Hg to about 60 mm/Hg.

Apart from maintaining general health of the eye, maintaining pressure in the eye may be important for eye procedures in general. For example, in addition to vitrectomy, other instruments such as scissors or forceps may be utilized to interact with or remove certain structural features within the eye. However, when the pressure dips too low, cutting, peeling or other direct interactions with these structural features may be inhibited. Even more concerning may be circumstances where pressure drops in the eye occur too quickly. For example, consider the fact that the surgeon may be directing an intervention at an eye feature with precision that requires millimeter, if not micrometer, level accuracy. Any sudden change in eye pressure may lead to a corresponding change in feature location.

Maintaining pressure with an infusion port device means that a tubular line running to the port is utilized to supply infusion fluid, or in some cases, air. Regardless, this means that a fixed port device may be secured to the preplaced cannula with the tubular line running across an exterior drape or surgical sheet in the area over the patient. Generally, this line is simply taped to the sheet to hold its position to avoid pulling on the infusion port. In addition to management of an insecure line, this is also done to help maintain the orientation of the infusion port. This prevents the port from angling too far away from perpendicular which could lead to the port interacting with choroid tissue at the interior of the eye and eventual blockage of the infusion function.

Of course, taping and re-taping of the infusion port line may be a time consuming and cumbersome task for the surgeon. This is particularly true in the case of eye surgery where there may be several occasions that call for the repositioning of the infusion port, for example to another preplaced cannula at another location. However, due to the significance of maintaining the infusion port properly oriented and unblocked with respect to the eye interior, any required orienting, taping and re-taping of the loose line will nevertheless be undertaken.

Similar circumstances may also be presented for other surgical implements utilized in eye surgery that are meant to be generally maintained in a fixed position. For example, this is often the case with light instruments. That is, in many cases the light instrument may be a hanging light instrument that is meant to be positioned through another preplaced cannula and immobilized in place. Orientation of the light instrument may be important during certain parts of the surgery. That is, once positioned for optimal lighting of the eye interior, movement of the light orientation may hamper visibility of the eye interior. Thus, to avoid this occurrence, an electric power line running from the light instrument may also be taped in place to help maintain light instrument orientation during eye surgery. Of course, just as with the infusion port, a variety of light positions may be utilized during the same surgery. Thus, taping and re-taping of the light instrument line may again be required.

Whether it be for the light instrument, the infusion port or any number of other fixed position devices, when it comes to eye surgery, the need for the surgeon to routinely tape and re-tape external lines or tubing may not simply be avoided. The surgeon is generally not free to simply allow such line and/or tubing to remain free. Thus, at present, surgeons are left with only the cumbersome option of taping and re-taping a variety of otherwise loose lines throughout most eye surgeries.

### SUMMARY

A surgical system for eye surgery is disclosed that utilizes a slotted tubing security platform. In one embodiment, the system includes a handheld instrument for performing a surgical procedure through a first preplaced cannula at an eye location. In some embodiments, the system also includes a stabilized instrument for reaching into the eye of the patient from a fixed position at a second preplaced cannula at another location of the eye. The slotted tubing security platform may be used to immobilize tubing coupled to the stabilized instrument to facilitate, for example, a substantially perpendicular orientation of the stabilized instrument relative the eye of the patient during the surgery.

### BRIEF DESCRIPTION OF THE DRAWINGS

FIG. 1 is perspective view of an embodiment of a slotted tubing security platform.

FIG. 2 is a top view of an eye of a patient during a draped surgery utilizing the slotted tubing security platform of FIG. 1.

FIG. 3 a side cross-sectional view of the eye during surgery such as depicted in FIG. 2 with multiple instruments of fixed orientation as aided by the platform of FIG. 1.

FIG. 4A is a perspective view of an alternate embodiment of a slotted tubing security platform.

FIG. 4B is a side view of another alternate embodiment of a slotted tubing security platform.

FIG. 4C is another side view of yet another alternate embodiment of a slotted tubing security platform.

FIG. 5 is a flow-chart summarizing an embodiment of employing a slotted tubing security platform to facilitate immobilization of an instrument during eye surgery.

### DETAILED DESCRIPTION

In the following description, numerous details are set forth to provide an understanding of the present disclosure. However, it will be understood by those skilled in the art that the embodiments described may be practiced without these particular details. Further, numerous variations or modifications may be employed which remain contemplated by the embodiments as specifically described.

Embodiments are described with reference to certain types of vitrectomy probe surgical procedures. In particular, a procedure in which vitreous humor is removed to address vitreous hemorrhage is illustrated. This, in turn, is accompanied by an infusion port that is to remain at a fixed location to maintain a tolerable pressure range within the eye during the procedure. However, tools and techniques detailed herein may be employed in a variety of other manners. For example, embodiments of a vitrectomy probe as detailed herein may be utilized to address retinal detachments, macular pucker, macular holes, vitreous floaters, diabetic retinopathy or a variety of other eye conditions. Regardless, so long as the procedure is accompanied by the use of a slotted tubing security platform to facilitate maintenance of an accompanying instrument and any associated line or tubing, appreciable benefit may be realized.

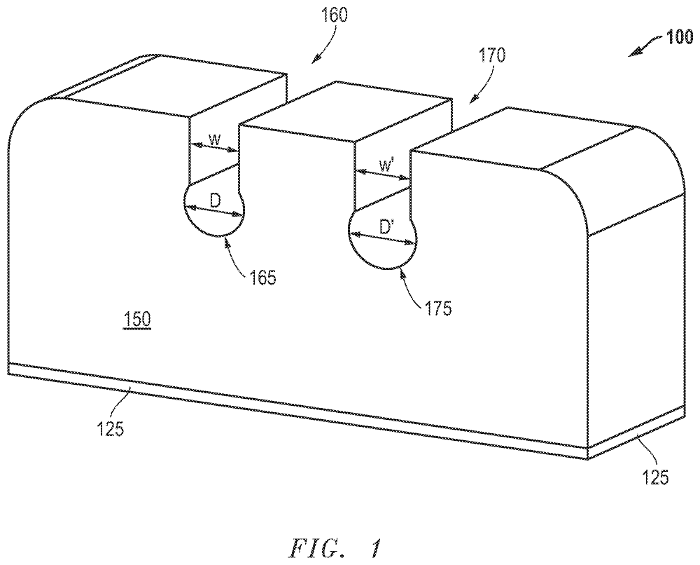

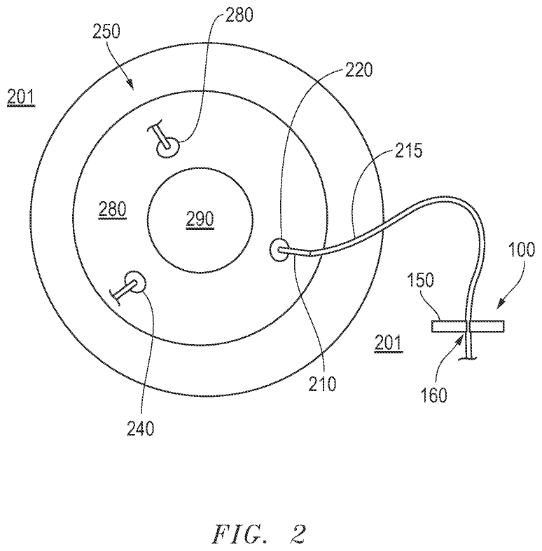

Referring now to FIG. 1, a perspective view of an embodiment of a slotted tubing security platform 100 is illustrated. With added reference to FIG. 2, the platform 100 is configured to immobilize tubing 215 running from a fixed position instrument 210 that is utilized as an aid to eye surgery (see eye 250). More specifically, the platform 100 includes multiple slots 160, 170 with accompanying orifices 165, 175 which may serve as locations for retaining tubing 215 of the instrument 210. For example, in one embodiment, the body 150 of the platform 100 may be of a die cut foam with the orifices 165, 175 sized to retain tubing 215 in a secure manner. The degree of force imparted on the tubing 215 may be sufficient for the retention without reaching a level prone to notably crimp or restrict any inner fluid flow of the tubing 215.

By way of example, in one embodiment the tubing 215 of FIG. 2 may range anywhere from 0.050 inches to about 0.095 inches in diameter with the diameter (D) of the corresponding orifice 165 being slightly smaller, perhaps about 5-10% smaller. Further, the body 150 may be 1-2 inches long with a profile less than about an inch. In an embodiment such as the one illustrated, where the platform 100 is of a die cut foam, the result may be a slight conformable squeezing manner of retention on the tubing 215. This may be sufficient to inhibiting movement of tubing 215 without creating any measurable form of restriction as noted above.

Continuing with reference to FIGS. 1 and 2, the platform 100 may firmly grasp the tubing 215 as suggested. However, the corresponding fixed nature of the platform 100 itself may further inhibit tubing movement. Thus, the body 150 of the platform 100 may be coupled to an adhesive 125 at its underside as illustrated in FIG. 1. In this way, the platform 100 may be adhered to a surgical drape or sheet 201 that is generally present for such surgical procedures, for example, to keep the eye 250 in a more isolated surgical environment. This means that with the securing of the tubing 215 by the platform 100, the also immobilized nature of the platform 100 at the sheet serves to inhibit tubing movement or keep the tubing fixed with relation to the patient. As detailed further below, this may be beneficial when it comes to ensuring a fixed, immobile orientation of the corresponding instrument 210 at the eye 250 during surgery.

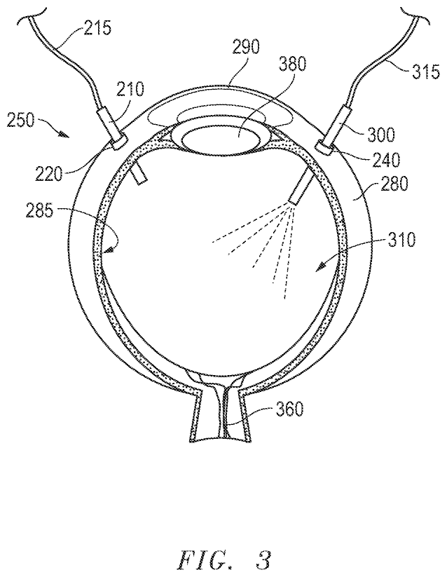

It is worth noting that the term “tubing” is not meant to infer that tubing 215 such as that of FIG. 2 must include a hollow or fluid-passable inner channel. For example, “tubing” may include a line such as a polymer jacket or other casing structure that is utilized in delivering electrical power to an instrument. Indeed, as described further below, the fixed instrument may be a hanging light instrument 300 with its own tubing or line 315 in the form of a power line that may benefit from immobilization with a platform 100 as described herein (see FIG. 3).

Referring specifically now to FIG. 2, a top view of an eye 250 of a patient is shown during a draped surgery utilizing the slotted tubing security platform 100 of FIG. 1. As with other minimally invasive procedures, a drape or sheet 201 of material is often found covering the patient in the area of the surgery with an opening to present the surgeon with a sanitary isolated region to which the surgery is directed. In this case, an eye 250 is presented. The eye 250 includes a centrally located cornea 290 and sclera 280. The delicate nature of the cornea 290 is such that intervention will generally be routed through the sclera 280 at offset locations. Specifically, in the embodiment shown, three separate preplaced cannulas 220, 240, 280 are illustrated at sclera locations. The cannulas 220, 240, 280 are of such minimal size and placed at the less delicate sclera 280 so as to minimize recovery time.

While embodiments herein are not limited to such scenarios as illustrated in FIG. 2, it is quite common for eye surgery to include three separate cannula placements as shown. In this way, one location is presented (280) through which a surgical tool may be presented for manipulation at an interior of the eye 250 while two others are also available, one for illumination (240) and another for an infusion port (220) (see also FIG. 3). Of course, additional locations may be included and in some cases fewer. However, so long as one cannula location 220, 240, 280 is configured to accommodate a fixed position instrument such as the infusion port 210, appreciable benefit of having a slotted tubing security platform 100 available may be realized.

As indicated, FIG. 2 illustrates the platform 100 adhered to the sheet 201 surrounding the surgical site. The immobilized platform 100 and body 150 may in turn be utilized to immobilize/inhibit movement of tubing 215 running from a fixed position instrument such as the infusion port 210. As shown, the tubing 215 is shaped to form a service loop between the platform 100 and the corresponding cannula 220. In this way the flexible tubing 215 is left tension-free between the stabilized cannula 220 and platform 100 locations. Thus, the tubing 215 is prevented from imparting any forcible movement onto the fixed port 210. As a result, a properly installed port 210, for example with a substantially perpendicular orientation relative the eye 250, is protected from any pull by the weight of the tubing that might otherwise affect port orientation. Once more, this is achieved without the requirement of the surgeon taping and re-taping the tubing 215 and forming the appropriate service loop with each taping and re-taping.

Avoiding the taping and re-taping steps may be particularly beneficial to the surgeon in the case of eye surgery. This is because instrument interchangeability is quite common for eye procedures. For example, in keeping with the illustrated scenario, the procedure might call for the infusion port 210 to be moved from the depicted location to the preplaced cannula at 240. However, with the availability of the platform 100, untapping and re-taping the tubing 215 is no longer necessary. Instead, the surgeon may disconnect the tubing 215 from the platform 100, relocate the port 210 to the adjacent cannula 240 and then re-stabilize the tubing 215 back at the platform 100 with a newly routed and shaped service loop in line with the new positioning. Thus, not only is the stabilizing of the tubing 215 more precise than the process of taping and re-taping, but the efficiency afforded to the surgeon in changing the placement is also enhanced.

Referring now to FIG. 3, a side cross-sectional view of the eye 250 during surgery such as depicted in FIG. 2 is shown with multiple instruments 210, 300 of fixed orientation as aided by the platform 100 of FIG. 1. For both instruments 210, 300 depicted, the fixed orientation is between about 45° off center from a vertical axis through the eye 250 (e.g., between about 45° and 135°). Further, the fixed nature means that a surgical procedure may proceed that is hands-free with respect to both instruments 210, 300. That is, for a properly installed and oriented infusion port 210 or light instrument 300, there may be no regular desire for repositioning or changing the depth of the intervention. Thus, so long as the associated tubing 215, 315 is contained, the surgery may proceed without any substantial further concern or attention regarding these instruments 215, 315. This is of significant benefit for the surgeon who may now focus primarily on a manual interventional tool such as a vitrectomy probe through another preplaced cannula (e.g., 280 of FIG. 2).

Continuing with reference to FIG. 3 and the example of a vitrectomy procedure, the interior 310 of the eye 250 is illuminated by the light instrument 300 which may be a conventional hanging light chandelier or other suitable fixed position device. Thus, a vitrectomy through another cannula (e.g., 280 of FIG. 2) may proceed in an illuminated manner, for example, avoiding retinal 360 or other more delicate features. The reliability of the illumination may be facilitated by the stable retention of the associated line 315 with the platform 100 of FIGS. 1 and 2.

In addition to reliably fixed illumination, pressure in the eye 250 may also be more reliably maintained by the infusion port 210. That is, due to the unique manner of reliable retention of the tubing 215, also with the platform 100, the stability of the orientation of the port 210 is more readily assured. As described above, this lessens the possibility of the port 210 becoming unintentionally angled too far away from perpendicular and becoming occluded by the choroid 285 surface defining the eye interior 310. Thus, sufficient pressure in the eye interior 310 may be maintained, for example, between about 15 and 60 mm/Hg. This is not only of benefit for eye health but may also help the surgeon carry out the vitrectomy or other more directly manual portion of the procedure. Sufficient pressure aids visualization, presents eye features to the surgeon in a more distinct manner and may facilitate peeling, cutting or other more manipulative interactions. Further, avoiding sudden pressure changes also helps to avoid unintentional eye 250 damage with the manual surgical implement due to sudden change in location of eye 250 features.

With added reference to FIG. 2, in addition to the benefit of maintaining a secure line 315 or tubing 215, the platform 100 provides a stable fixed reusable device. Thus, when a procedure such as illustrated in FIG. 3 calls for instrument change, for example, exchanging the light instrument 300 at one cannula 240 with the infusion port 210 at the other 220, this may efficiently be accomplished without imprecise taping and re-taping. Instead, the surgeon may simply dislodge the line 315 and tubing 215 from the platform 100 if necessary and reposition thereat with new service looping that fits the new locations of the instruments 300, 210.

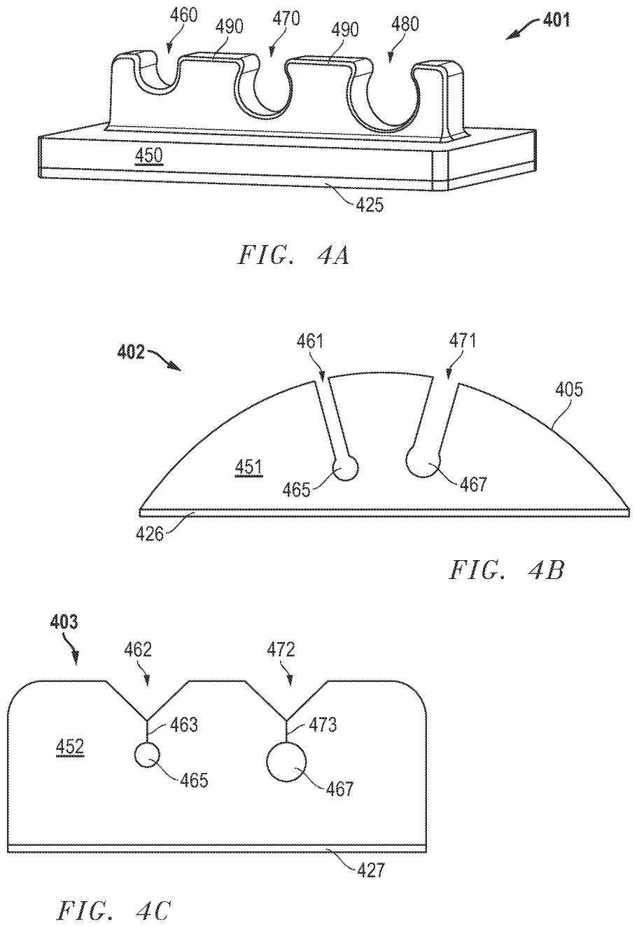

Referring now to FIGS. 4A-4C, alternate embodiments of slotted tubing security platforms 401, 402, 403 are illustrated. For the embodiment of FIG. 4A, the body 450 may be rigid injection molded plastic. An adhesive layer 425 would again be provided as detailed above. However, for this embodiment, retention in the orifices 460, 470, 480 would not be by way of foam compression. Instead, the illustrated orifices 460, 470, 480 serve as slots that are also partially defined by raised structures 490 that lead to a snap fit of different tubing sizes.

With specific reference to FIG. 4B, the platform 402 again includes a body 451 and adhesive layer 426. In this embodiment, the body 451 is also of a curved outer surface 405 to expand the amount of surface presented from the surgeon's perspective. Thus, reaching a given slot 461 or 471 for directing tubing to an orifice 465 or 467 may be achieved in a more ergonomically accessible manner. Once more, for the illustrated embodiment, the orifices 465, 467 and slots 461, 471 may be differently sized depending on likely tubing sizes retained. Along these lines, the body 451 may be a die cut foam with the orifices 465, 467 and slots 461, 471 sized slightly smaller than expected tubing sizes as detailed above.

For the platform 403 embodiment of FIG. 4C, another die cut foam body 452 may be utilized with an underlying adhesive layer 427. In this instance, the slots 462, 472 may be concave for ergonomic locating of closed slits 463, 473 that may be traversed by tubing in reaching orifices 465, 467 below. Of course, the slits 463, 473 involve two foam sides making contact that may be separated as the tubing traverses to an orifice 465, 467. However, as also described above, each orifice 465, 467 may be 5-10% smaller in diameter than the diameter of the tubing to be retained by the platform 403 in a compressible fashion that does not materially restrict any flow through the tubing. Alternatively, in yet another embodiment, the body 452 may be orifice-free with the slots 462, 472 terminating at the slits 463, 473, which directly serve to achieve the tubing retention.

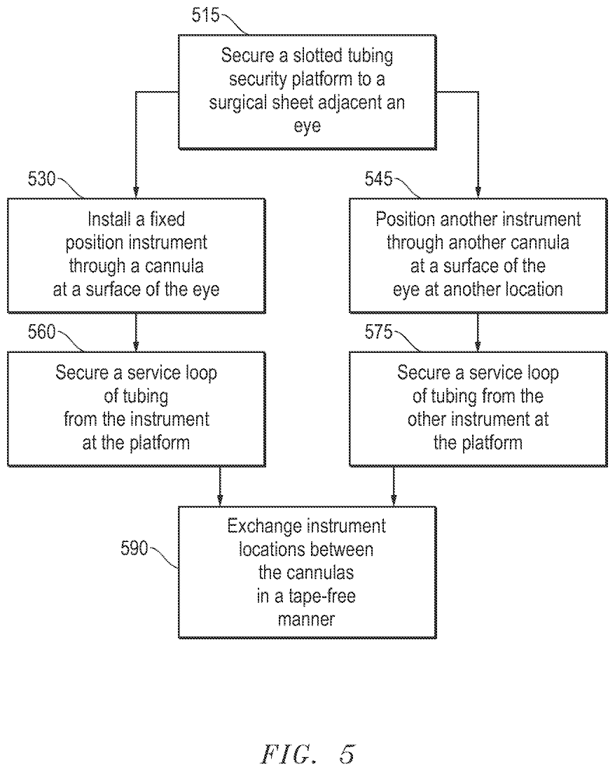

Referring now to FIG. 5, a flow-chart is shown summarizing an embodiment of employing a slotted tubing security platform to facilitate immobilization of an instrument during eye surgery. Specifically, as noted at 515, the platform is secured to a surgical sheet adjacent an eye. A fixed position instrument is then installed through a cannula at an eye surface (see 530). As indicated at 560, a service loop of tubing from the installed instrument may be secured at the platform. Similarly, as indicated at 545, another instrument may be positioned through another cannula at a surface of the eye. This may be a manual or otherwise mobile interventional device or, as noted at 575, another device for installation where another service loop of tubing is secured at the platform. Regardless, as indicated at 590, instrument locations may efficiently be exchanged in a tape-free manner by removing and resecuring instrument tubing at the platform as needed.

Embodiments described hereinabove include unique devices and techniques for avoiding taping and re-taping service loops running from fixed position surgical devices. This is of particular benefit when it comes to an infusion port utilized during eye surgery for the reasons detailed above. However, it is also of benefit for other fixed position surgical devices such as light instruments or even devices that are utilized outside of eye surgery.

The preceding description has been presented with reference to presently preferred embodiments. However, other embodiments and/or features of the embodiments disclosed but not detailed hereinabove may be employed. Furthermore, persons skilled in the art and technology to which these embodiments pertain will appreciate that still other alterations and changes in the described structures and methods of operation may be practiced without meaningfully departing from the principle and scope of these embodiments. Additionally, the foregoing description should not be read as pertaining only to the precise structures described and shown in the accompanying drawings, but rather should be read as consistent with and as support for the following claims, which are to have their fullest and fairest scope.

We claim:

1. A slotted tubing security platform to facilitate surgery, the platform comprising:
a body with at least one slot for accommodating tubing running from a surgical device at a fixed surgical position of a patient; and
an adhesive mechanism coupled to the body for securing the platform to a surgical sheet adjacent the surgical position.

2. The platform of claim 1 wherein the slot is a channel to an orifice for the accommodating.

3. The platform of claim 2 wherein the orifice is of a diameter smaller than an outer diameter of the tubing.

4. The platform of claim 3 wherein the body is of a die cut foam for compressible retention of the tubing at the orifice.

5. The platform of claim 1 wherein the body is of a die cut foam and the slot is a guide to a slit for compressible retention of the tubing at the orifice.

6. The platform of claim 1 wherein the body is of a rigid molded configuration for snap fit retention of the tubing.

7. A surgical system for eye surgery, the system comprising:
a handheld instrument for performing a surgical procedure in an eye of a patient through a first preplaced cannula at an eye location;
a stabilized instrument for reaching into the eye of the patient from a fixed position at a second preplaced cannula at another location of the eye; and
a slotted tubing security platform to inhibit tubing movement of tubing coupled to the stabilized instrument to facilitate a fixed orientation of the stabilized instrument relative the eye of the patient.

8. The surgical system of claim 7 wherein the tubing is one of a fluid line and a power line.

9. The surgical system of claim 7 wherein the fixed orientation is between about 45° and 135° relative the eye of the patient.

10. The surgical system of claim 9 wherein the fixed orientation is substantially perpendicular relative the eye of the patient.

11. The surgical system of claim 7 wherein the stabilized instrument is one of an infusion port and a light instrument.

12. A method of performing eye surgery, the method comprising:
securing a slotted tubing security platform to a surgical sheet adjacent an eye;
installing a fixed position instrument through a cannula at a surface of the eye; and
securing a service loop of tubing from the instrument at the platform.

13. The method of claim 12 further comprising positioning another instrument through another cannula at a surface of the eye at another location.

14. The method of claim 13 further comprising one of:
securing another service loop of tubing from the other instrument at the platform; and
performing a manual surgical procedure with the other instrument.

15. The method of claim 13 further comprising:
exchanging instrument locations between the cannulas; and
resecuring service loops of the tubings at the platform.
